(19) United States
(12) Patent Application Publication
(10) Pub. No.: US 2004/0018224 A1
(43) Pub. Date: Jan. 29, 2004

(54) Supplementation of the diet with cholesterol results in a large stimulation of the plasma omega-3 fatty acid content

(76) Inventors: Grant Pierce, Manitoba (CA); James Gilchrist, Manitoba (CA); Anton Lukas, Manitoba (CA)

Correspondence Address: Michael R.Williams, Ade & Company, 1700-360 Main Street, Winnipeg, MB, R3C 3Z3, CA

(21) Appl. No.: 10/622,538

(22) Filed: Jul. 21, 2003

Related U.S. Application Data
(60) Provisional application No. 60/397,679, filed on Jul. 23, 2002.

(51) Int. Cl. A61K 31/56; A61K 47/00; A61K 31/202

(52) U.S. Cl. 424/439; 514/171; 514/560

(57) ABSTRACT

A combination and a method for producing edible animal products with elevated levels of omega-3 fatty acids is described.

This application claims priority under 35 USC § 119(e) to Provisional Patent Application Serial No. 60/397,679 filed on Jul. 23, 2003.

### FIELD OF THE INVENTION

The present invention relates generally to the fields of animal feed, feed supplements and food products.

### BACKGROUND OF THE INVENTION

Omega-3 fatty acids are thought to have important beneficial effects as a therapeutic strategy against cardiovascular disease, arthritis and cancer. Omega-3 fatty acids are also known to promote production of prostaglandins and leukotrienes of the N-3 series (PGE₃, LTB₅, etc) and reduce production from the N-6 series (PGE₂, LTB₅ etc), which are pro-inflammatory. It is particularly important for the diet of growing children. If we could find more efficient ways to increase the plasma concentrations of omega-3 fatty acids, it would provide potent health-related benefits.

To this end, many companies are marketing omega-3 fatty acid to the human population as an oil supplement or in capsule form. In addition, omega-3 enriched eggs and milk products are now on the market or are currently under development to provide alternative methods for the human population to ingest therapeutic doses of omega-3 fatty acids. To achieve these results in the animal products, animals are fed fatty acid enriched products (fishmeal or flaxseed supplementation to their diets) and this is metabolically processed by the animal to increase the omega-3 fatty acid content of their milk or eggs. This method typically results, in some experiments, in a 5 fold increase, which is not entirely satisfactory. Clearly, methods which produce a greater increase in the omega-3 fatty acid content are needed, as these would be of greater benefit.

### SUMMARY OF THE INVENTION

According to a first aspect of the invention, there is provided a pharmaceutical composition comprising: a source of omega-3 fatty acids; and cholesterol.

According to a second aspect of the invention, there is provided an animal feed comprising: a source of omega-3 fatty acids; and cholesterol.

According to a third aspect of the invention, there is provided a method of producing an edible animal product with elevated levels of omega-3 fatty acids comprising: producing an edible animal product having elevated levels of omega-3 fatty acids by feeding the animal a feed comprising a source of omega-3 fatty acids and cholesterol; and collecting the edible animal product.

According to a fourth aspect of the invention, there is provided an egg having elevated levels of omega-3 fatty acids prepared as described above.

According to a fifth aspect of the invention, there is provided milk having elevated levels of omega-3 fatty acids prepared according to the method described above.

According to a sixth aspect of the invention, there is provided a method of increasing uptake of omega-3 fatty acids in a patient in need thereof, comprising: administering to a patient in need thereof cholesterol and a source of omega-3 fatty acids.

According to a seventh aspect of the invention, there is provided a kit comprising: cholesterol; and a source of omega-3 fatty acids.

### BRIEF DESCRIPTION OF THE DRAWINGS

FIG. 1—Total plasmid lipids over 8 weeks.

FIG. 2—Plasma fatty acid profile of rabbits at end of 8 week study.

FIG. 3—Plasma fatty acid profile of rabbits at start of 8 week study.

FIG. 4—Plasma fatty acid profile of rabbits at end of 8 week study.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Definitions

Herein, flaxseed oil is used as the exemplary source of omega-3 fatty acids. As will be appreciated by one of skill in the art, other suitable sources of omega-3 fatty acids are well-known in the art and include for example, but are by no means limited to flaxseed, soy, linseed oil, sea plankton, and deep-sea cold water fatty fish (e.g. tuna, salmon, sardine, mackerel).

As used herein, “animal product” refers to edible animal products, for example, meat, milk and eggs.

As used herein, “effective amount” refers to the administration of an amount of a given compound or composition that achieves the desired effect.

As used herein, the term “treating” in its various grammatical forms refers to preventing, curing, reversing, attenuating, alleviating, minimizing, suppressing or halting the deleterious effects of a disease state, disease progression, disease causitive agent or other abnormal condition.

As used herein, “autoimmune disease” refers to diseases wherein the host immune system recognizes “self” material as foreign. Examples include but are by no means limited to rheumatoid arthritis, lupus erythrematosis, multiple sclerosis, inflammatory bowel diseases, for example, Crohn's disease and ulcerative colitis, autoimmune hemolytic anemia, autoimmune thrombocytopenic purpura, autoimmune hepatitis and pancreatitis, Goodpasture's syndrome, acute rheumatic fever, pemphigus vulgaris, myasthenia gravis, ankylosing spondylitis, acute anterior uveitis, Grave's disease, Hashimoto's thyroiditis and juvenile diabetes.

As used herein, cholesterol is one of the exemplary components. However, other structurally similar compounds may be substituted for cholesterol within the invention, for example, but by no means limited to sterols, phytosterols and other detergent-like compounds.

As used herein, “animal” refers to vertebrates.

Described herein is a method of supplementing animal feed with omega-3 fatty acids in combination with cholesterol which increases the omega-3 concentration in animal products derived from animals fed the animal feed. Also described is a method of promoting omega-3 fatty acid production in humans either in need or desirous of such treatment comprising a pharmaceutical composition comprising a mixture of cholesterol and a source of omega-3 fatty acids.

In one embodiment of the invention, an animal feed, prepared according to means known in the art, is supplemented with a source of omega-3 fatty acids and cholesterol. The feed may be supplemented by: adding the cholesterol and source of omega-3 fatty acids during preparation of the feed; applying the cholesterol and source of omega-3 fatty acids as a coating to the animal feed; or including the cholesterol and source of omega-3 fatty acids separately, for example, adding pellets comprising cholesterol and the source of omega-3 fatty acids to traditional feed pellets.

As will be appreciated by one of skill in the art, the relative concentrations of the source of omega-3 fatty acids and cholesterol will vary according to the specific animal feed being prepared, the animal for which it is intended, the size, condition and age of the animal and the desired result. For example, in some embodiments, animal feed is supplemented with 0.1-25%, or in some embodiments, preferably 0.5-15% flaxseed and 0.1-5% cholesterol (w/w).

Specifically, in some embodiments, feeds are produced for egg-producing fowl, for example, chickens and turkeys. As will be appreciated by one of skill in the art, supplementing traditional fowl feed(s) known in the art with cholesterol and a source of omega-3 fatty acids will result in the production of edible animal products, for example, eggs, having a much higher content of omega-3 fatty acids.

In yet other embodiments, feed for milk-producing animals, for example, cows and goats, are supplemented with cholesterol and a source of omega-3 fatty acids at concentrations discussed above. As will be appreciated by one of skill in the art, feeding the animals a traditional feed known in the art supplemented as described above will result in the production of milk having elevated omega-3 fatty acid content.

In yet other embodiments, feed for meat-producing animals, for example, cattle, swine, chickens, turkeys and the like, are supplemented with cholesterol and a source of omega-3 fatty acids at concentrations discussed above. As will be appreciated by one of skill in the art, feeding the animals a traditional feed known in the art supplemented as described above will result in the production of meat and meat by-products having elevated omega-3 fatty acid content.

As will be apparent to one of skill in the art, using the above-described methods, wherein a traditional animal feed is supplemented with a source of omega-3 fatty acids and cholesterol at concentrations discussed above, fed to an animal and the animal product is collected and/or processed using means known in the art, will result in edible animal products which have elevated levels of omega-3 fatty acids.

As discussed above, the diseases for which omega-3 fatty acids are known or believed to comprise effective treatments for slowing disease progression, reducing severity of symptoms, and preventing contraction of the disease include for example, cardiovascular disease, arthritis, cancer, other autoimmune diseases, for example, lupus erythrematosis, multiple sclerosis, inflammatory bowel diseases, for example, Crohn's disease and ulcerative colitis, autoimmune hemolytic anemia, autoimmune thrombocytopenic purpura, autoimmune hepatitis and pancreatitis, Goodpasture's syndrome, acute rheumatic fever, pemphigus vulgaris, myasthenia gravis, ankylosing spondylitis, acute anterior uveitis, Grave's disease, Hashimoto's thyroiditis and juvenile diabetes, depression, headaches, ADHD, as well as other similar diseases known in the art.

Thus, individuals suffering from or at risk of developing one or more of the diseases listed above or other diseases known in the art to be alleviated, ameliorated or prevented by omega-3 fatty acids or individuals simply interested in better overall health will purchase these edible animal products for consumption as part of their diet.

In yet other embodiments, an animal feed supplemented with cholesterol and a source of omega-3 fatty acids at concentrations discussed above may be prepared for consumption by animals, for example, household pets or livestock. As will be appreciated by one of skill in the art, administering these feeds to the animals will result in better overall health for the animals, preventing contraction of disease, for example, shipping fever.

In yet other embodiments, there is provided a pharmaceutical composition comprising a source of omega-3 fatty acids and cholesterol. As will be appreciated by one of skill in the art, the concentration of the source of omega-3 fatty acids and cholesterol may vary according to many factors, including, for example, dosage schedule, condition being treated, as well as the condition and weight of the patient. Specifically, the combination of cholesterol and the source of the omega-3 fatty acids improves the efficiency of uptake and the absorption of the omega-3 fatty acids, meaning that lower overall concentrations of omega-3 fatty acids are needed to have a beneficial effect.

In some embodiments, the pharmaceutical composition comprising a source of omega-3 fatty acids in admixture with cholesterol may be combined with other compounds or compositions known in the art such that the composition is in the form of, for example, a pill, tablet, liquid, film or coating using means known in the art and as discussed below.

In some embodiments, the above-described pharmaceutical composition at concentrations or dosages discussed above may be combined with a pharmaceutically or pharmacologically acceptable carrier, excipient or diluent, either biodegradable or non-biodegradable. Exemplary examples of carriers include, but are by no means limited to, for example, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, mixtures thereof and the like. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches. See, for example, Remington: The Science and Practice of Pharmacy, 1995, Gennaro ed.

As will be apparent to one knowledgeable in the art, specific carriers and carrier combinations known in the art may be selected based on their properties and release characteristics in view of the intended use. Specifically, the carrier may be pH-sensitive, thermo-sensitive, thermo-gelling, arranged for sustained release or a quick burst. In some embodiments, carriers of different classes may be used in combination for multiple effects, for example, a quick burst followed by sustained release.

In other embodiments, the above-described pharmaceutical composition at concentrations or dosages described above may be encapsulated for delivery. Specifically, the pharmaceutical composition may be encapsulated in biodegradable microspheres, microcapsules, microparticles, or nanospheres. The delivery vehicles may be composed of, for example, hyaluronic acid, polyethylene glycol, poly(lactic acid), gelatin, poly(E-caprolactone), or a poly(lactic-glycolic) acid polymer. Combinations may also be used, as, for example, gelatin nanospheres may be coated with a polymer of poly(lactic-glycolic) acid. As will be apparent to one knowledgeable in the art, these and other suitable delivery vehicles may be prepared according to protocols known in the art and utilized for delivery.

The invention provides kits for carrying out the methods of the invention. Accordingly, a variety of kits are provided, including for example, a pharmaceutical composition of cholesterol and a source of omega-3 fatty acids as described above or separate pharmaceutical compounds of cholesterol and a source of omega-3 fatty acids, prepared as described above.

The kits of the invention comprise one or more containers comprising a pharmaceutical composition of cholesterol and a source of omega-3 fatty acids as described above or separate pharmaceutical compounds of cholesterol and a source of omega-3 fatty acids, a suitable excipient as described herein and a set of instructions, generally written instructions although electronic storage media (e.g., magnetic diskette or optical disk) containing instructions are also acceptable, relating to the use and dosage of the pharmaceuticals for the intended treatment. The instructions included with the kit generally include information as to dosage, dosing schedule, and route of administration for the intended treatment. The containers may be unit doses, bulk packages (e.g., multi-dose packages) or sub-unit doses.

The invention will now be described by way of examples. However, the invention is not limited to the examples.

### EXAMPLE 1

Male New Zealand White rabbits were fed a control rabbit chow (RG), a 10% ground flaxseed supplemented diet (FX), a 0.5% cholesterol supplemented diet (OL) or a 0.5% cholesterol supplemented diet including 10% flaxseed (CF). Rabbits were fed the diet over 8 weeks and plasma lipid composition was evaluated regularly by gas chromatography analysis.

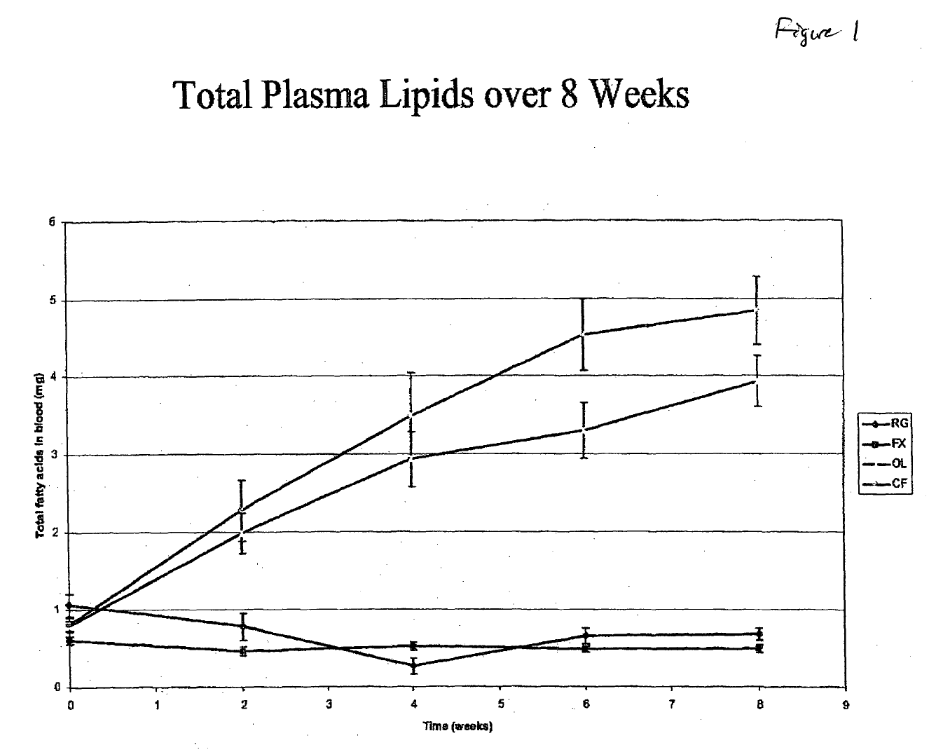

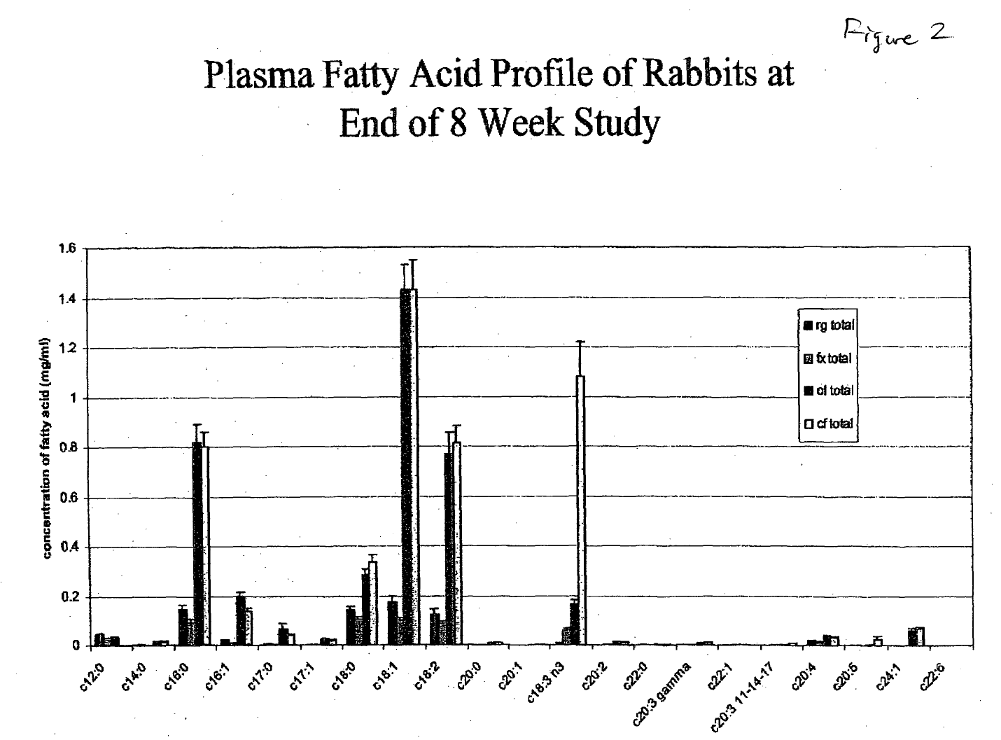

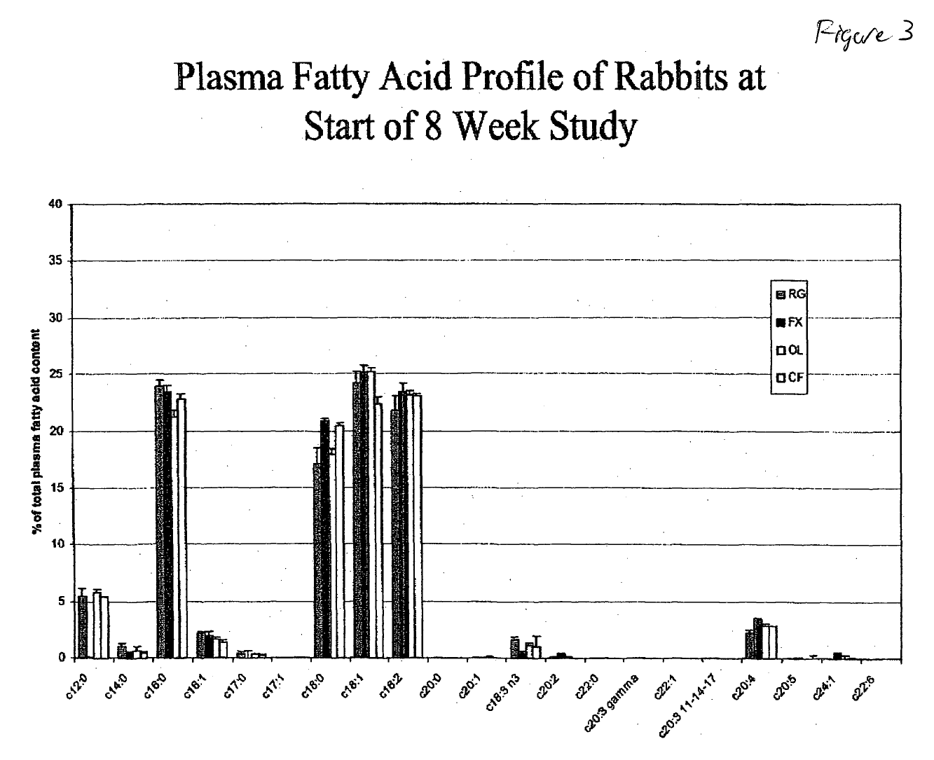

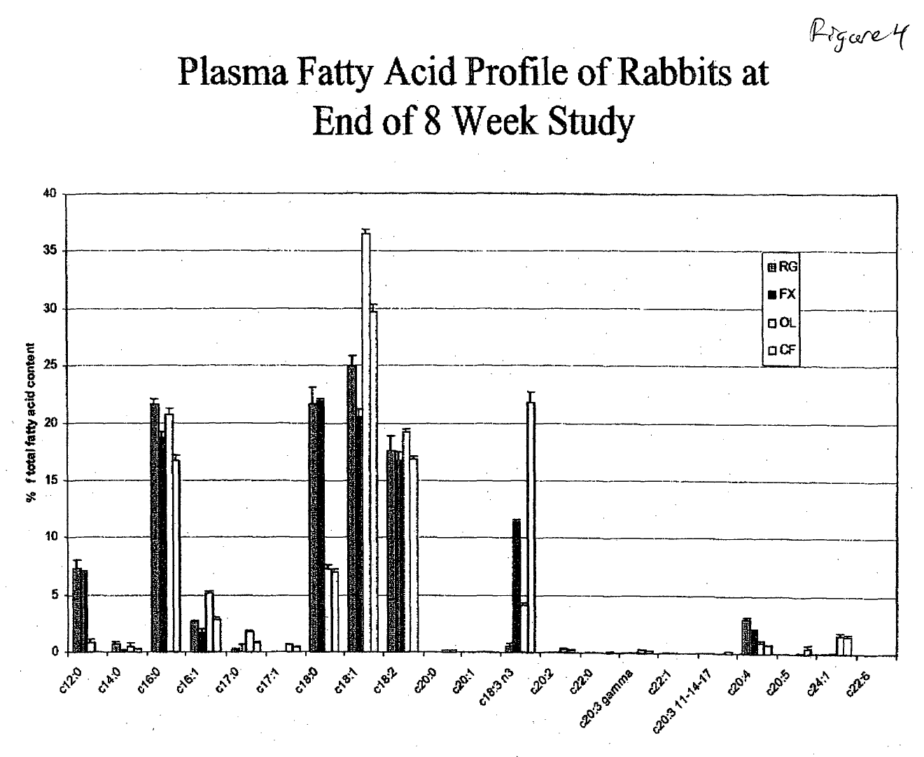

The results demonstrate that total fatty acid content of the blood is elevated strongly in the CF or OL group, as shown in FIG. 1. As shown in FIG. 2, these increases were due primarily to large changes in 16:0, 18:1, 18:2 and 18:3 fatty acids. We are primarily interested in the 18:3 omega-3 fatty acid. This fatty acid increases in concentration from less than 1% of all fatty acids (FIGS. 3 and 4) to greater than 20% in the CF group (FIG. 4).

Supplementation of the diet with cholesterol in the presence of a source of omega-3 fatty acids therefore results in an abnormally large stimulation of omega-3 levels in the blood.

While the preferred embodiments of the invention have been described above, it will be recognized and understood that various modifications may be made therein, and the appended claims are intended to cover all such modifications which may fall within the spirit and scope of the invention.

1. A pharmaceutical composition comprising:
a source of omega-3 fatty acids; and
cholesterol.

2. The pharmaceutical composition according to claim 1 wherein the source of omega-3 fatty acids is flaxseed.

3. An animal feed comprising:
a source of omega-3 fatty acids; and
cholesterol.

4. A method of producing an edible animal product with elevated levels of omega-3 fatty acids comprising:
producing an edible animal product having elevated levels of omega-3 fatty acids by feeding the animal a feed comprising a source of omega-3 fatty acids and cholesterol; and
collecting the edible animal product.

5. The method according to claim 4 wherein the animal is a fowl and the edible animal product is an egg.

6. The method according to claim 4 wherein the edible animal product is milk.

7. An egg having elevated levels of omega-3 fatty acids prepared according to the method of claim 5.

8. Milk having elevated levels of omega-3 fatty acids prepared according to the method of claim 6.

9. A method of increasing uptake of omega-3 fatty acids in a patient in need thereof, comprising:
administering to a patient in need thereof cholesterol and a source of omega-3 fatty acids.

10. A kit comprising:
cholesterol; and
a source of omega-3 fatty acids.
